(19) United States
(12) Patent Application Publication
(10) Pub. No.: US 2002/0076393 A1
(43) Pub. Date: Jun. 20, 2002

(54) Method for stimulation of defensin production

(75) Inventors: Pascale Fehlbaum, Ambler, PA (US); Mark Anderson, Norristown, PA (US); Meena Rao, Monmouth Junction, NJ (US); Michael Zasloff, Merion Station, PA (US)

Correspondence Address: MORGAN, LEWIS & BOCKIUS, 1800 M STREET NW, WASHINGTON, DC, 20036-5869, US

(73) Assignee: MAGAININ PHARMACEUTICALS, INC.

(21) Appl. No.: 09/935,785

(22) Filed: Aug. 24, 2001

Related U.S. Application Data
(63) Continuation of application No. 09/316,386, filed on May 21, 1999, now abandoned.
(60) Provisional application No. 60/086,275, filed on May 21, 1998.

(51) Int. Cl. A61K 38/19; A61K 31/198

(52) U.S. Cl. 424/85.1; 514/561

(57) ABSTRACT

The subject invention relates to a method for the stimulation of defensin production in eukaryotic cells such as, for example, mammalian cells. Furthermore, the invention includes said method for the prevention and treatment of infections and other various disease states and in the stimulation of the immune system.

### CROSS REFERENCE TO RELATED APPLICATIONS

This application claims priority to U.S. Provisional Application Ser. No. 60/086,275, filed May 21, 1998, which is herein incorporated by reference in its entirety.

### FIELD OF THE INVENTION

This invention relates to stimulating the production of defensins in mammalian cells using the amino acid isoleucine or active isomers or analogs thereof. Furthermore, the present invention includes the use of isoleucine or active isomers or analogs thereof to stimulate defensins for the prevention and treatment of infections and other various disease states.

### BACKGROUND OF THE INVENTION

Defensins are cationic, cysteine-rich peptides that display broad spectrum antimicrobial activity. Their structure is characterized by a conserved cysteine motif that forms three disulfide linkages, imposing a characteristic β-sheet structure (Hill et al., 1991; White et al., 1995). Associated with this structure is an amphiphilic charge distribution that enables the defensins to interact with and disrupt target cell membranes (Lehrer et al., 1989). This disruption is thought to be accomplished by the formation of channels in the target membrane, leading to cell lysis (Kagan et al., 1990). Defensins have been shown to inhibit proliferation of both gram-positive and gram-negative bacteria, yeast and numerous viruses. In particular, defensins inhibit the proliferation of the yeast strain Candida albicans and the gram-negative bacteria Escherichia coli (Porter et al., 1997; Harder et al., 1997; Schonwetter et al., 1995; Daher et al., 1986).

Defensins have recently been identified as an integral component of the antimicrobial barrier of mucosal surfaces. In both the human and murine small intestine, defensin RNA has been localized to the Paneth cell, a specialized epithelial cell located at the crypt base (Ouellette et al., 1989; Jones et al., 1992). The associated peptide has been localized within secretory granules of the Paneth cell and in the lumen of the small intestine, suggesting a role for defensins in host defense in the gut (Selsted et al., 1992). Defensins have also been found in bovine and human respiratory epithelium. Tracheal antimicrobial peptide, a β-defensin isolated from bovine tracheal mucosa, was localized to the ciliated columnar epithelial cells of the trachea and bronchi (Diamond et al., 1991; Diamond et al., 1993). Lingual antimicrobial peptide, another β-defensin, was found in bovine lingual mucosa and stratified squamous epithelium of the tongue (Schonwetter et al., 1995). Most recently, human β-defensin-1 was demonstrated to be present in the epithelium of the trachea and bronchi, as well as the submucosal gland and alveolar epithelium (Goldman et al., 1997; Zhao et al., 1996).

Considerable data exists indicating that epithelial defensins are up-regulated in response to infection. In cultured tracheal epithelial cells, tracheal antimicrobial peptide message is induced following exposure to bacterial lipopolysaccharide (Diamond et al., 1996). This induction was blocked by antibody to CD14, suggesting that epithelial cells provide an active, inducible antimicrobial defense. Following injury to bovine tongue, lingual antimicrobial peptide RNA message increased at the site of injury (Schonwetter et al., 1995). Induction of lingual antimicrobial peptide was also observed following acute infection in bronchial epithelium and chronic infection in ileal mucosa (Stolzenberg et al., 1997). Together these data support a role for β-defensins as important host defense effector molecules that are rapidly mobilized by epithelium upon injury or infection.

Due to the significant host defense properties of defensins, any means which stimulates or induces the production of these peptides is desired in the art. The present invention provides such means as to stimulate the production of defensins.

### SUMMARY OF THE INVENTION

The present invention comprises a method of increasing the production of defensins in eukaryotic cells. This method comprises exposing the eukaryotic cells to a composition comprising isoleucine or active isomers or analogs thereof in an amount sufficient to effect said increase. The method also comprises increasing defensin production in eukaryotic cells using isomers of isoleucine including stereoisomers, diastereomers in particular or a combination thereof. The stereoisomers include L-isoleucine, D-isoleucine and D-allo-isoleucine. The method further comprises increasing defensin production in eukaryotic cells using active analogs of isoleucine including alpha-keto-methyl-valerate, isoleucine hydroxamate, butyrate, and valine.

The eukaryotic cells where defensin production may be stimulated may be mammalian cells, and more particularly, epithelial cells. These epithelial cells may be from a tissue or source selected from, for example, the group comprising brain, kidney, heart, spleen, buccal mucosa, nasal mucosa, conjunctiva, tongue, choroid plexus, trachea, bronchi, bronchioles, fallopian tubes, uterus, cervix, vagina, testes, bladder, urethra, esophagus, duodenum, jejunum, ileum, caecum, ascending colon, sigmoid colon, descending colon and rectum.

Furthermore, the invention includes a method of treating or preventing an infection or other disease state in a patient. This method comprises administering a composition comprising isoleucine or active isomers or analogs thereof in an amount sufficient to effect treatment or prevention. The infection may be caused by any viral, bacterial or fungal pathogen including, for example, Candida albicans, Escherichia coli, Rotavirus or Respiratory Syncytial Virus. The invention also encompasses a method of stimulating the immune system of a mammal comprising administering to the mammal, isoleucine or active isomers or analogs thereof in an amount sufficient to effect the stimulation.

The accompanying figures, which are incorporated in and constitute a part of this specification, illustrate several embodiments of the invention and together with the description, serve to explain the principle of the invention.

### BRIEF DESCRIPTION OF THE FIGURES

FIG. 1: Effect of various amino acids on defensin production in MDBK cells.

FIG. 2: Dose-response effect of L-isoleucine on defensin production in MDBK cells.

FIG. 3: Dose-response effect of D-isoleucine on defensin production in MDBK cells.

FIG. 4: Comparison of effects of isoleucine and alloisoleucine on defensin production in MDBK cells.

FIG. 5: Effect of L-isoleucine on defensin production in the human colon epithelial cell line HT-29.

FIG. 6: Defensin inducing effect of alpha-keto-methylvalerate.

FIG. 7: Defensin inducing effect of butyrate.

FIG. 8: Defensin inducing effect of isoleucine hydroxamate.

FIG. 9: Defensin inducing effect of valine.

FIG. 10: Illustration of generic structure of defensin inducing isoleucine analogs.

### DETAILED DESCRIPTION OF THE INVENTION

Definitions

“Consisting essentially of” herein refers to compositions wherein the named chemical constituent, active isomer or analog thereof is the principal ingredient of said composition.

“Active isomer” herein refers to molecules having the same molecular formula of a named chemical constituent but differing in the nature or sequence of binding of their atoms or in the spatial arrangement of their atoms, wherein said molecules elicit defensin production. “Analogs” as used herein refers to molecules having the generic or similar structure of a named chemical constituent (e.g., corresponding α-keto form of a named amino acid), wherein said “analogs” elicit defensin production.

The subject invention relates to a method of stimulating the production of defensins in eukaryotic cells using a composition comprising isoleucine or active isomers or analogs thereof. The composition used in the method may be used to prevent or treat various disease states or conditions. Consequently, a method of treating or preventing an infection or other disease state in a patient may comprise administering a composition comprising isoleucine or active isomers or analogs thereof, in an amount sufficient to effect the treatment or prevention of said infection or disease state.

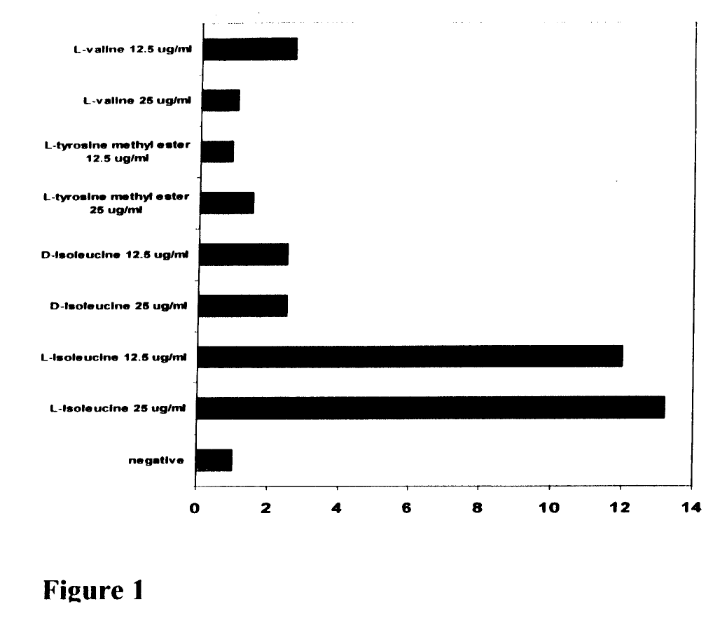

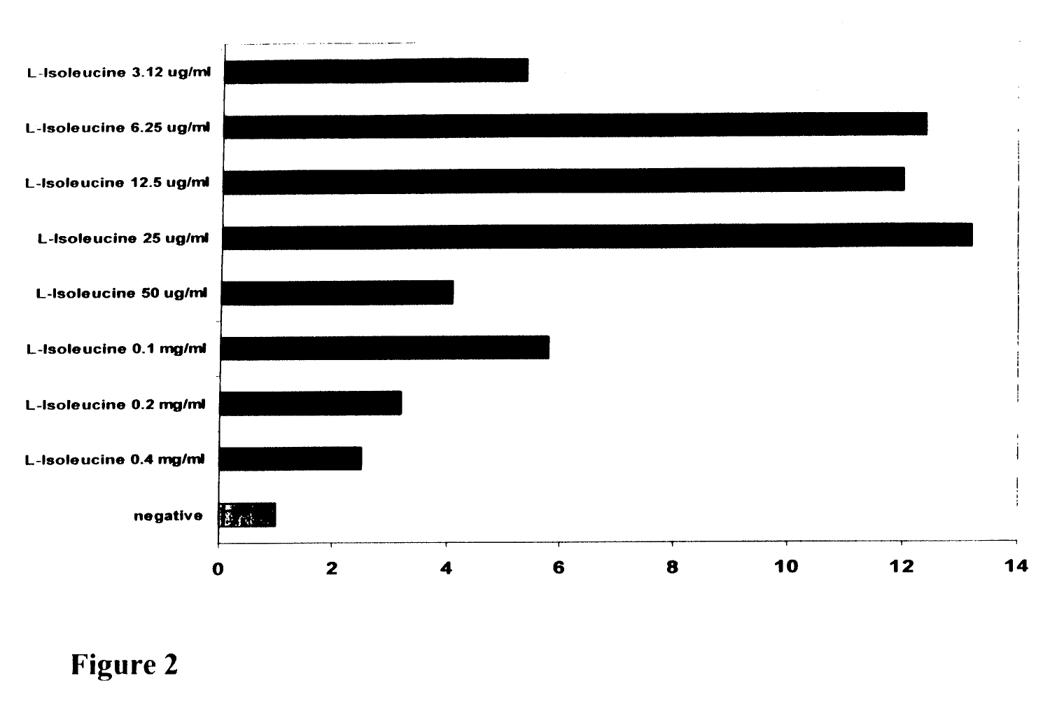

As exemplified in FIG. 1, L-isoleucine has the ability to stimulate the production of defensins in eukaryotic cells. L-isoleucine was capable of stimulating defensin production by MDBK cells at concentrations as low as three micrograms per milliliter. None of the other similar amino acids tested at this concentration had any effect on defensin production. L-valine and L-tyrosine methyl ester had no effect on defensin production at concentrations as high as fifty micrograms per milliliter. Furthermore, L-isoleucine increased defensin production at concentrations fifty to one-hundred-fold less than those of D-isoleucine, indicating that the desired effect of the method is dependent on the stereochemical configuration of isoleucine. FIG. 2 demonstrates the dose-dependent effect of L-isoleucine on defensin production, indicating the specific effect of L-isoleucine. Thus, the present invention encompasses a method of eliciting the production of defensins by eukaryotic cells. This method comprises exposing the cells to the composition containing isoleucine or active isomers or analogs thereof in an amount sufficient to elicit the production of defensins by the cells.

In one embodiment, isoleucine or active isomers or analogs are exposed to cells ex vivo at concentrations which elicit the highest production of defensin. Preferably, cells are exposed to concentrations of L-isoleucine from about 3.12 μg/ml to about 100 μg/ml, more preferably from about 6.25 μg/ml to about 50 μg/ml, even more preferably from about 6.25 μg/ml to about 25 μg/ml. Further, cells can be exposed to D-isoleucine at concentrations which elicit the production of defensin. Preferably, cells are exposed to concentrations of D-isoleucine from about 100 μg/ml to about 400 μg/ml, more preferably from about 200 μg/ml to about 400 μg/ml.

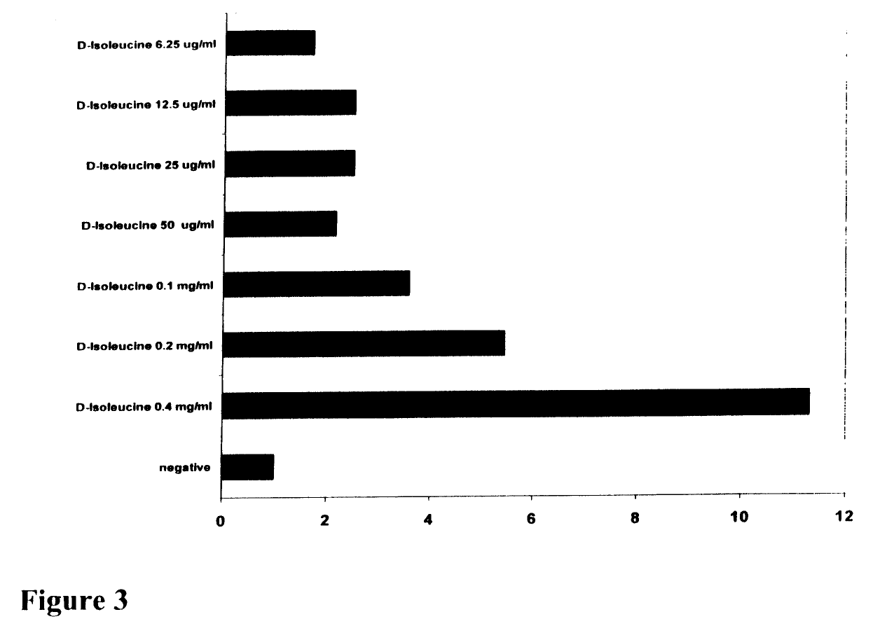

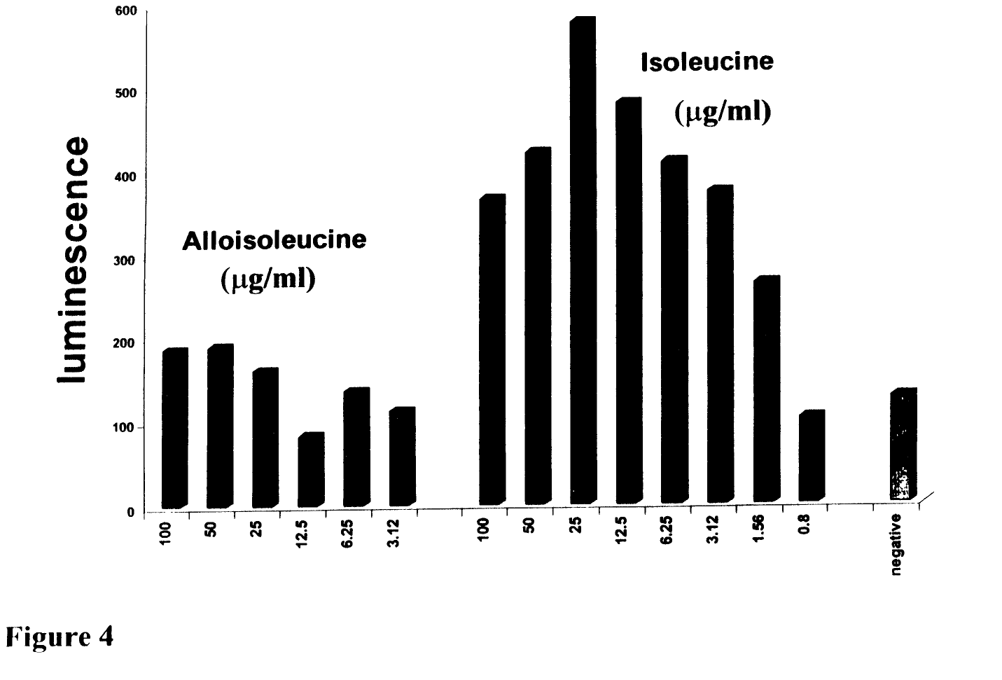

The method of the invention encompasses a composition comprising isoleucine or active isomers thereof. Isomers of isoleucine comprise both stereoisomers and diastereomers as isoleucine has two chiral centers allowing for four separate stereoisomers. The importance of stereochemistry to the present invention will be apparent to one skilled in the art because L-isoleucine stimulated defensin production at concentrations approximately fifty to one-hundred-fold less than D-isoleucine as exemplified in FIGS. 2 and 3. Similarly, changing the configuration of isoleucine at its second chiral center yields the compound alloisoleucine. FIG. 4 shows that alloisoleucine is not as effective an inducer of defensin production in MDBK cells as is isoleucine. The strong dependence of defensin inducing activity on the chiral configuration of isoleucine supports the specificity of isoleucine as a defensin inducer. Thus, the present method of the invention comprises a method whereby the composition for stimulating defensin production contains L-isoleucine, D-isoleucine, D-alloisoleucine, or a mixture thereof.

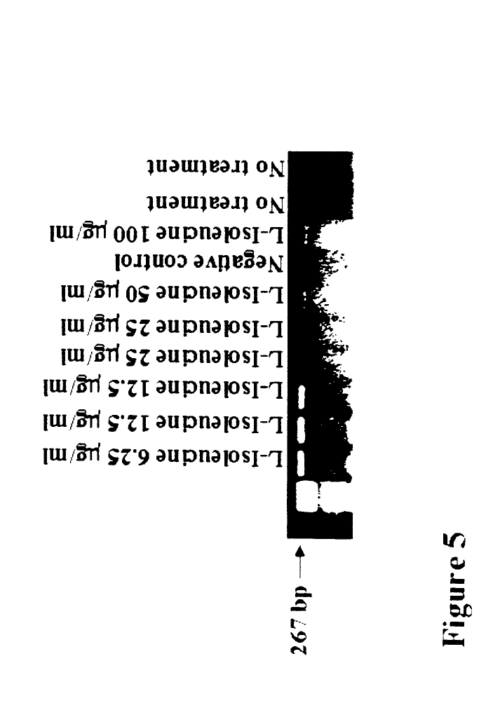

FIG. 5 illustrates the defensin inducing property of L-isoleucine in the human colon epithelial cell line HT-29. This result, taken together with similar data from MDBK cells shows that isoleucine has utility as a defensin inducer in a variety of species and at a variety of epithelial surfaces that may be of therapeutic importance.

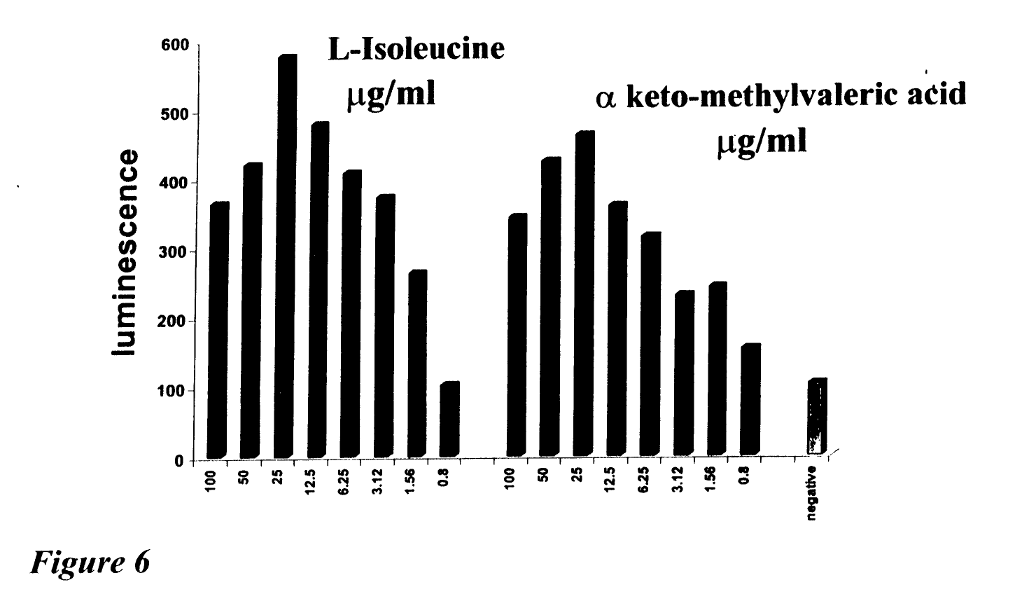

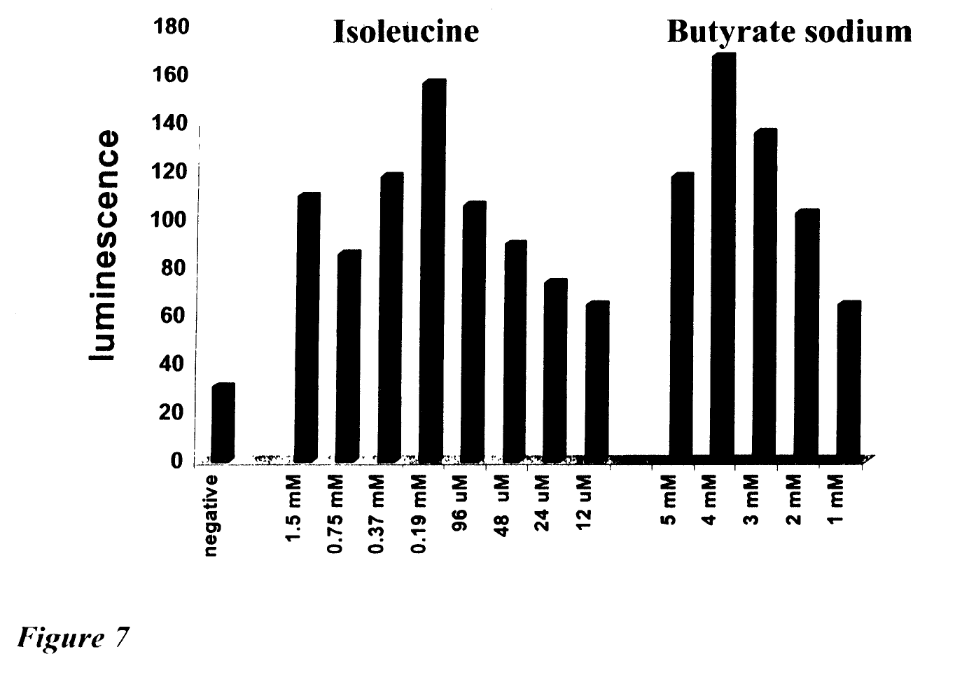

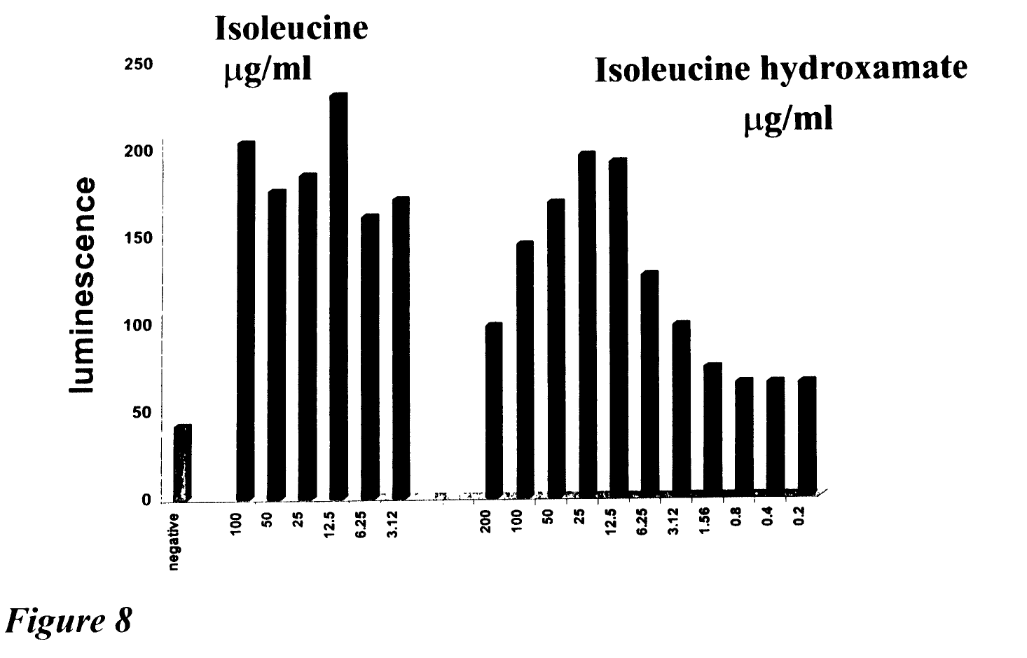

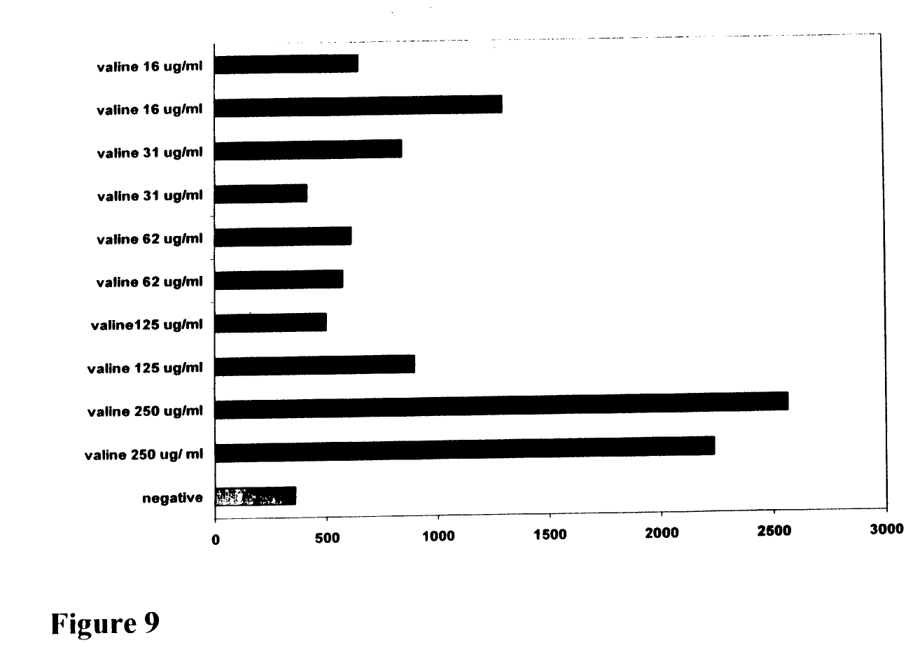

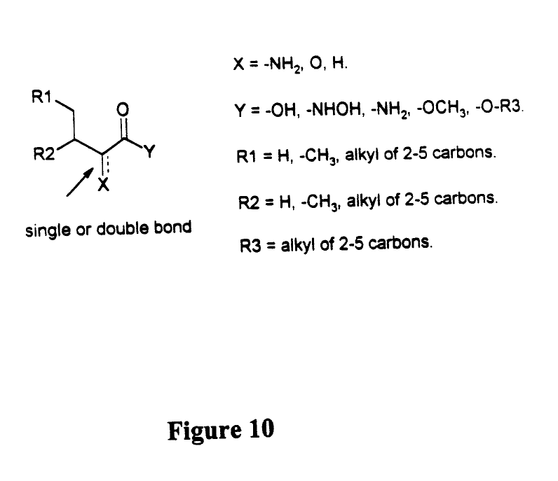

In addition to L-isoleucine and D-isoleucine other similar molecules also act as defensin inducers. FIGS. 6, 7, and 8 demonstrate that the compounds alpha-keto-methylvalerate, butyrate, and isoleucine hydroxamate are inducers of defensin production in epithelial cells. FIG. 9 shows that valine stimulates defensin expression at concentrations that are substantially higher than those needed for isoleucine but which may have utility in treating or preventing infection. In a preferred embodiment, FIG. 10 illustrates a generic structure for defensin inducing isoleucine analogs.

The method of the present invention comprises stimulation of defensin production by epithelial cells derived from, for example, the following mammalian tissues: brain, skin, kidney, heart, spleen, buccal mucosa, nasal mucosa, conjunctiva, tongue, choroid plexus, trachea, bronchi, brochioles, fallopian tubes, uterus, cervix, vagina, testes, bladder, urethra, esophagus, duodenum, jejunum, ileum, caecum, ascending colon, sigmoid colon, descending colon and rectum. The method may also be used to stimulate defensin production in epithelial cells found in other tissues, for example, the ear, liver, pancreas or ovary. For example, the method may be utilized for the treatment or prevention of dermal, oral, ocular, respiratory, gastrointestinal, colorectal and urogenitary diseases or other epithelial cell-related diseases in mammals, including humans and animals.

The method of the invention is useful in treating or preventing infections resulting from a broad range of pathogens as defensins have been shown to inhibit proliferation of both gram-positive and gram-negative bacteria, yeast and numerous viruses. For example, the method is effective in the treatment of candidiasis because defensins inhibit the proliferation of the underlying pathogen Candida albicans. The method is also be useful in treating diarrhea, dysentery, septicemia and acute infantile gastroenteritis as defensins are known to inhibit proliferation of the underlying pathogens of these disease states, particularly Escherichia coli. Treatment of acute respiratory disease resulting from Respiratory Syncytial Virus will also be possible as defensins inhibit the proliferation of such viruses.

The present invention also includes a method of stimulating the immune system of a mammal after, for example, surgery, immune ablation by chemotherapy or other treatments, or bacterial or viral infections. Such a method comprises administering a composition comprising isoleucine or active isomers or analogs thereof to the patient, human or animal, requiring immune system stimulation in an amount sufficient to effect such stimulation. Thus, stimulation of defensin production in the epithelial cells of the patient is the mechanism whereby stimulation of the immune system occurs. Further methods of the invention inlcude methods which comprise immunostimulation by administering compositions comprising isoleucine or active isomers or analogs thereof and cytokines or other immune stimulants. Administration of such compositions are preferred for diseases involving gram-negative infections and septic conditions wherein LPS (lipopolysaccharide) is the major immunogenic agent. In a preferred embodiment, administration can be sequential or more preferably simultaneous (i.e., co-administered).

The invention includes pharmaceutical compositions comprising isoleucine or active isomers or analogs thereof or a combination of isoleucine or active isomers or analogs thereof, together with a pharmaceutically acceptable carrier. Preferred embodiments may include such compositions comprising purified isoleucine, active isomers or analogs or combinations of any of the mentioned compounds. Acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical carriers are described in Remington's Pharmaceutical Sciences, 19th edition, Mack Publishing Company, 1995. The pharmaceutical compositions used in the method of treatment of this invention may be administered systemically or topically, depending on such considerations as the condition to be treated, need for site-specific treatment, quantity to be administered and similar considerations. This administration may be by the oral, intravenous, or inhaled route or by suppository, enema, mouth wash or the like.

Topical administration may be used. Any common topical formation such as a solution, suspension, gel, ointment or salve and the like may be employed. Preparation of such topical formulations are well described in the art of pharmaceutical formulations as exemplified, for example, by Remington's Pharmaceutical Sciences, 19th edition, Mack Publishing Company, 1995. For topical application, these compounds could also be administered as a powder or spray, particularly in aerosol form or as a lozenge for local oral delivery. The active ingredient may be administered in pharmaceutical compositions adapted for systemic administration. As is known, if a drug is to be administered systemically, it may be confected as a powder, pill, tablet or the like or as a syrup or elixir for oral administration. For intravenous, intraperitoneal or intralesional administration, the compound will be prepared as a solution or suspension capable of being administered by injection. In certain cases, it may be useful to formulate these compounds in suppository form or as an extended release formulation for deposit under the skin or intramuscular injection.

An effective amount is that amount which will increase the expression of defensins. A given effective amount will vary from condition to condition and in certain instances may vary with the severity of the condition being treated and the patient's susceptibility to treatment. Accordingly, a given effective amount will be best determined at the time and place through routine experimentation as shown in FIG. 2. However, it is anticipated that in the treatment and prevention of infections and other disease states is in accordance with the present invention, a formulation containing between 0.001 and 5.0% by weight, preferably about 0.01 to 1.0%, will usually constitute an effective therapeutic amount. When administered systemically, an amount between 0.01 and 100 milligrams per kilogram body weight per day, but preferably about 0.1 to 10 milligrams per kilogram, will effect a therapeutic result in most instances.

The present compositions are preferably for treatment of human subjects, however, the mentioned compositions are also contemplated for use in animal subjects, including farm animals as well as domestic species.

The practice of the present invention will employ the conventional terms and techniques of molecular biology, pharmacology, immunology and biochemistry that are within the ordinary skill of those in the art. For example, see Sambrook et al., Molecular Cloning: A Laboratory Manual, 3rd edition, Cold Spring Harbor Laboratory Press, 1989.

Other embodiments of the invention will be apparent to those skilled in the art from consideration of the specification and practice of the invention disclosed. It is intended that the specifications and examples be considered exemplary only with the true scope of the invention being indicated by the claims. Having provided this detailed information, applicants now describe preferred aspects of the invention.

### EXAMPLE 1

### Defensin Induction in MDBK Cells

Cell Culture: MDBK (Madin-Darby Bovine Kidney) cells were obtained from the ATCC (Rockville, Md.) and were maintained in growth medium consisting of Eagle's modified essential media with Earle's balanced salt solution, 10% horse serum, 0.10 mM non-essential amino acids and no antibiotics. For stimulation experiments, cells were plated into six well tissue culture plates and maintained for three days in growth medium until cells were almost confluent. The medium was then changed to serum-free epithelial cell growth medium (Clonetics, San Diego, Calif.) and the test material was added to the dish. Twenty-four hours later, the medium was withdrawn and cells were rinsed with phosphate-buffered saline. Total RNA was then isolated using Trizol reagent (Gibco/BRL, Grand Island, N.Y.) according to protocols supplied by the manufacturer. RNA was quantified by measuring the OD₂₆₀ of each sample.

RNA-Polymerase Chain Reaction: Total RNA was treated with DNase prior to reverse transcription and PCR. The DNase was heat inactivated at 65° C. in the presence of EDTA for ten minutes. Reverse transcription and PCR were performed essentially as described for the GeneAmp RNA PCR kit (Perkin Elmer, Foster City, Calif.). Briefly, approximately 250 nanograms of total RNA was primed with polydT and reverse transcribed with Murine Leukemia Virus reverse transcriptase in a total volume of 16 μL at room temperature for ten minutes and then at 42° C. for an additional fifteen minutes. The reverse transcriptase was heat inactivated at 99° C. for five minutes and the reaction was chilled to 4 degrees C. This reverse transcription reaction was then split in half: one portion was used for amplification of the target defensin RNA, and the other was treated in parallel to determine the β-tubulin RNA level as a control. Additional reagents necessary for the PCR reaction, including appropriate synthetic DNA primers;

| | | |
|---|---|---|
| β-defensin | 5′ CTC TTC CTG GTC CTG TCT 3′ | (SEQ ID NO: 1) |
| | 5′ CTT CTT TTA CTT CCT CGT GCA GCA 3′ | (SEQ ID NO: 2) |
| β-tubulin | 5′ GTT CCC AAA GAT GTC AAT GCT GCC 3 | (SEQ ID NO: 3) |
| | 5′ ATG CTG CAA GGC TGA AAG GAA TGG 3′ | (SEQ ID NO: 4) |

were added after splitting the reverse transcription reactions to bring the reaction volumes to 40 μL. The reactions were then subjected to thermal cycling as follows: 95° C. for one minute, 52° C. for one minute, 72° C. for one minute for 30 cycles followed by a single 72° C. incubation for fifteen minutes to allow for extension. The expected 200 base pair β-defensin PCR product was measured by gel electrophoresis or QPCR (Quantitative PCR).

Product Capture for QPCR System: 10 μL of the final PCR mixture was combined with 15 μL of strepavidin bead slurry (Perkin-Elmer, Foster City, Calif.), 21 μL H₂O and 4 μL of 10× PCR buffer to yield a 50 μL binding reaction. The binding reactions were incubated at room temperature for fifteen minutes with occasional agitation. One ml of QPCR assay buffer was then added to the reaction. The quantity of PCR product was subsequently measured with the Perkin-Elmer QPCR instrument (Perkin-Elmer, Foster City, Calif.). The amount of defensin product was normalized to endogenous expression in MDBK cells in the absence of any experimental agent.

### EXAMPLE 2

### Defensin Induction in MDBK Cells

In order to easily identify compounds that have defensin inducing activity a stable cell line containing an integrated plasmid in which expression of the easily assayed gene product luciferase is controlled by a bovine beta-defensin promoter was constructed.

Cloning of bovine defensin promoter: A DNA fragment containing the bovine enteric beta-defensin (EBD) promoter was generated via PCR. The fragment contained 812 base pairs of 5′-flanking sequence and the first 43 base pairs of the 5′ untranslated portion of the EBD cDNA. This DNA fragment was engineered to contain an Mlu I restriction site at the 5′ end of the fragment and a Bgl II restriction site at the 3′ end to facilitate subsequent cloning into the pGL2-basic luciferase expression plasmid (Promega). The PCR product was cloned into the TA cloning vector (Invitrogen) by standard techniques and sequenced to confirm its identity.

Construction of EBD promoter-luciferase reporter plasmid: The defensin promoter containing TA-vector plasmid was digested with Mlu I and Bgl II and the appropriate digestion product was isolated following separation on a 1.2% agarose gel. The luciferase expression vector pGL2-basic was similarly digested with Mlu I and Bgl II and isolated following gel electrophoresis. The vector and defensin promoter fragment were ligated together and transformed into E. coli via standard procedures.

Generation of Stable MDBK cell lines containing a functional defensin promoter-luciferase plasmid: The defensin promoter-luciferase plasmid was mixed with the G418 resistance plasmid LNCZ in a 1 to 5 ratio, combined with Fugene™ transfection reagent and placed on MDBK cells.

Cells were then exposed to medium containing 0.4 milligrams/ml G418 until resistant colonies were visible (4-5 weeks). The resistant clones were then expanded and screened for the expression of luciferase.

Screening of compounds for defensin inducing activity: Cells of a clonal MDBK cell line expressing the defensin promoter-luciferase plasmid were placed in the wells of a 96-well tissue culture plate. Test substances were then placed in tissue culture medium and added to the wells. After the test substances had been in contact with the cells for 12-24 hours luciferase expression levels were measured using standard procedures.

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. All references, patents or other publications cited in this application or in the following reference section are herein incorporated by reference in their entirety.

### REFERENCES

Daher K A, Selsted M E and Lehrer R I. Direct inactivation of viruses by human granulocyte defensins. J. Virol. 60, 1068-1074, 1986.

Diamond G, Zasloff M, Eck H, Brasseur M, Maloy W L and Bevins C L. Tracheal antimicrobial peptide, a cysteine-rich peptide from mammalian tracheal mucosa: peptide isolation and cloning of a cDNA. Proc. Natl. Acad. Sci. USA 88, 3952-3956, 1991.

Diamond G, Jones D E and Bevins C L. Airway epithelial cells are the site of expression of a mammalian antimicrobial peptide gene. Proc. Natl. Acad. Sci. USA 90, 4596-4600, 1993.

Diamond G, Russell J P and Bevins C L. Inducible expression of an antibiotic peptide gene in lipopolysaccharide-challenged tracheal epithelial cells. Proc. Natl. Acad. Sci. USA 93, 5156-5160, 1996.

Goldman M J, Anderson G M, Stolzenberg E D, Kari U P, Zasloff M and Wilson J M. Human beta-defensin-1 is a salt-sensitive antibiotic in lung that is inactivated in cystic fibrosis. Cell 88, 553-560, 1997.

Harder J, Bartels J, Christophers E and Schröder J M. A peptide antibiotic from human skin. Nature 387, 861, 1997.

Hill C P, Yee J, Selsted M E and Eisenberg D. Crystal structure of defensin HNP-3, an amphiphilic dimer: mechanisms of membrane permeabilization. Science 251, 1481-1485, 1991.

Jones D E and Bevins C L. Paneth cells of the human small intestine express an antimicrobial peptide gene. J. Biol. Chem. 267, 23216-23225, 1992.

Lehrer R I, Barton A, Daher K A, Harwig S S, Ganz T and Selsted M E. Interaction of human defensins with Escherichia coli. Mechanism of bactericidal activity. J. Clin. Invest. 84, 553-561, 1989.

Ouellette A J, Greco R M, James M, Frederick D, Naftilan J and Fallon J T. Developmental regulation of cryptdin, a corticostatin/defensin precursor mRNA in mouse small intestinal crypt epithelium. J. Cell Biol. 108, 1687-1695, 1989.

Porter E M, Van Dam E, Valore E V and Ganz T. Broad-spectrum antimicrobial activity of human intestinal defensin five. Infect. Immun. 65, 2396-2401, 1997.

Kagan B L, Selsted M E, Ganz T and Lehrer R I. Antimicrobial defensin peptides form voltage-dependent ion-permeable channels in planar lipid bilayer membranes. Proc. Natl. Acad. Sci. USA 87, 210-214, 1990.

Schonwetter B S, Stolzenberg E D and Zasloff M. Epithelial antibiotics induced at sites of inflammation. Science 267, 1645-1648, 1995.

Selsted M E, Miller S I, Henschen A H and Ouellette A J. Enteric defensins: antibiotic peptide components of intestinal host defense. J. Cell Biol. 118, 929-936, 1992.

Stolzenberg E D, Anderson G M, Ackerman M R, Whitlock R H and Zasloff M. Epithelial antibiotic induced states of disease. Proc. Natl. Acad. Sci. USA 94, 8686-8690, 1997.

White S H, Wimley W C, and Selsted M E. Structure, function, and membrane integration of defensins. Curr. Opin Struct. Biol. 5, 521-527, 1995.

Zhao C, Wang I and Lehrer R I. Widespread expression of beta-defensin hBD-1 in human secretory glands and epithelial cells. FEBS Lett. 396, 319-322, 1996.

What is claimed:

1. A method of eliciting the production of defensins in eukaryotic cells comprising exposing said cells to a composition comprising isoleucine or active isomers or analogs thereof in an amount sufficient to elicit said production.

2. The method of claim 1, wherein said composition consists essentially of isoleucine or active isomers or analogs thereof in an amount sufficient to elicit said production.

3. The method of claim 1, wherein said composition further comprises a cytokine.

4. The method of any one of claims 1-3, wherein said eukaryotic cells are mammalian cells.

5. The method of claim 4, wherein said mammalian cells are epithelial cells.

6. The method of claim 5, wherein said epithelial cells are from a tissue or source selected from the group consisting of brain, kidney, heart, spleen, buccal mucosa, nasal mucosa, conjunctiva, tongue, choroid plexus, trachea, bronchi, bronchioles, fallopian tubes, uterus, cervix, vagina, testes, bladder, urethra, esophagus, duodenum, jejunum, ileum, caecum, ascending colon, sigmoid colon, descending colon and rectum.

7. A method according to any one of claims 1-3, wherein the isomers are stereoisomers.

8. A method according to claim 7, wherein the stereoisomer is L-isoleucine.

9. A method according to claim 7, wherein the stereoisomer is D-isoleucine.

10. A method according to claim 7, wherein the stereoisomer is D-alloisoleucine.

11. A method according to any one of claims 1-3, wherein the analog is alpha-keto-methylvalerate.

12. A method according to any one of claims 1-3, wherein the analog is isoleucine hydroxamate.

13. A method according to any one of claims 1-3, wherein the analog is valine.

14. A method according to any one of claims 1-3, wherein the analog is butyrate or an active derivative thereof.

15. A method according to any one of claims 1-4, wherein the analog is one of a member of a class of compounds defined by the following chemical structure:

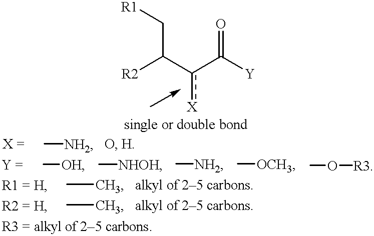

16. A method of treating or preventing an infection or other disease state in a patient in need of said treatment or prevention by administering to said patient a composition consisting essentially of isoleucine or active isomers or analogs thereof in an amount sufficient to effect the treatment or prevention.

17. The method of claim 16, wherein said infection is caused by any viral, bacterial or fungal pathogen.

18. The method of claim 13, wherein the pathogen is selected from the group consisting of Candida albicans, Escherichia coli, Rotavirus or Respiratory Syncytial Virus.

19. A method of stimulating the immune system of a mammal or other animal comprising administering to said mammal or other animal a composition comprising isoleucine or active isomers or analogs thereof in an amount sufficient to effect said stimulation.

20. The method of claim 16, wherein the isoleucine or active isomer or analog thereof is administered after surgery, immune ablation or bacterial or viral infection.
